(19) United States
(12) Patent Application Publication
(10) Pub. No.: US 2020/0276518 A1
(43) Pub. Date: Sep. 3, 2020

(54) COMPONENT SEPARATION METHOD USING SUPERCRITICAL FLUID CHROMATOGRAPH

(71) Applicant: Shimadzu Corporation, Kyoto-shi, Kyoto (JP)

(72) Inventor: Jana RYKL, Reinach (CH)

(73) Assignee: Shimadzu Corporation, Kyoto-shi, Kyoto (JP)

(21) Appl. No.: 16/646,274

(22) PCT Filed: Nov. 6, 2017
(86) PCT No.: PCT/JP2017/039933

§ 371 Date: Mar. 11, 2020

(51) Int. Cl. B01D 15/40; B01D 15/42; C07K 1/16

(52) U.S. Cl. CPC B01D 15/40; C07K 1/16; B01D 15/426

(57) ABSTRACT

For the separation of a peptide or protein in a sample using a supercritical fluid chromatograph, carbon dioxide is used as the supercritical fluid, while an organic solvent containing hexafluoroisopropanol is used as the modifier to be added to the supercritical fluid. If a mixed liquid of methanol, isopropanol, water and formic acid is simply used as the modifier, a considerable amount of peak tailing occurs on the chromatogram, causing the peak waveform to be distorted. By comparison, when a mixed liquid prepared by additionally mixing hexafluoroisopropanol in the mixed liquid of methanol, isopropanol, water and formic acid is used, the peak observed on the chromatogram will have a sharp, satisfactory shape.

### TECHNICAL FIELD

The present invention relates to a method for separating components in a sample using a supercritical fluid chromatograph, and more specifically, to a component separation method suitable for separating highly polar components in a sample for the purpose of an analysis or purification.

### BACKGROUND ART

A supercritical fluid chromatograph (which may hereinafter be abbreviated as the “SFC”) is a type of chromatograph in which a supercritical fluid, i.e. a fluid whose temperature and pressure exceed their respective critical points (critical temperature and critical pressure), is used as the primary mobile phase. It has the characteristic that its eluting power can be controlled by regulating the temperature and pressure of the mobile phase. In the SFC, carbon dioxide is popularly used as the supercritical fluid due to its comparatively low critical points in both temperature and pressure, easy-to-handle nature, inexpensiveness, and other reasons. The SFC using carbon dioxide as the supercritical fluid has the advantage that the low viscosity of the fluid allows the mobile phase to be supplied at a higher velocity to complete the analysis within a shorter period of time than conventional high-performance liquid chromatographs (HPLC; see Patent Literature 1, Non Patent Literature 1 or other documents).

Carbon dioxide in the supercritical state has approximately the same degree of hy-drophobicity as hexane. In many cases, carbon dioxide as singly used cannot elute the target component from the column. Therefore, an organic solvent, called the “modifier”, is normally added to the supercritical fluid to regulate the polarity of the mobile phase so that a wide variety of compounds including not only hydrophobic compounds but also hydrophilic compounds can be analyzed. As the modifier, an organic solvent in which carbon dioxide is miscible is commonly used, such as methanol, ethanol, isopropyl alcohol or acetonitrile. For an analysis of a highly polar compound, an organic compound to which an acid (e.g. formic acid or acetic acid), salt (e.g. ammonium formate or ammonium acetate) or base (e.g. diethylamine) is added is used as the modifier.

### CITATION LIST

### Patent Literature

Patent Literature 1: US 2016/0202218 A1

### Non Patent Literature

Non Patent Literature 1: “Nexera UC Supercritical Fluid Extraction/Chromatograph System”, Shimadzu Corporation, [online], [accessed on Jun. 26, 2017], the Internet <URL: http://www.shimadzu.com/an/hplc/nexera_uc/index.html>

### SUMMARY OF INVENTION

### Technical Problem

In recent years, analysis of peptides or proteins using liquid chromatograph mass spectrometers (LC-MS) have been actively performed. Meanwhile, there has also been a strong demand for an analysis of peptides or proteins using an SFC from the viewpoints of the speed-up of the analysis, improvement of the separation characteristics or other favorable effects. However, using the conventional combination of the supercritical fluid and modifier as the mobile phase restricts the polarity and molecular weight of the components that can be satisfactorily separated. Therefore, it has been difficult to satisfactorily separate highly polar, high-molecular-weight components, such as peptides or proteins.

The present invention has been developed in view of the previously described problem. Its objective is to provide a component separation method using a supercritical fluid chromatograph, by which highly polar, high-molecular-weight components, such as peptides or proteins, can be separated with a high level of separation performance.

### Solution to Problem

The present invention developed for solving the previously described problem is a component separation method using a supercritical fluid chromatograph, including the steps of injecting a sample into a mobile phase containing a supercritical fluid and a modifier, and introducing the mobile phase with the sample into a column so as to make components in the sample separate from each other while passing through the column, the method characterized in that:

carbon dioxide is used as the supercritical fluid, and the modifier contains hexafluoroisopropanol.

Hexafluoroisopropanol is a polar substance and exhibits strong hydrogen-bonding characteristics. Therefore, it can act as a highly effective solvent to a compound including a group which acts as a hydrogen-bond acceptor, such as the amide group or amino group. Besides, hexafluoroisopropanol is an optically transparent substance within the ultraviolet wavelength region and will not significantly inhibit the ionization of the target component in the case where the supercritical fluid chromatograph is combined with a mass spectrometer including an electrospray ion source. Therefore, adding hexafluoroisopropanol to the mobile phase in the supercritical fluid chromatograph will barely produce adverse effects on the component detection.

An experimental study by the present inventor has confirmed that peptides can be detected with a high level of separation performance independently of their concen-trations by using a mobile phase prepared by adding a modifier containing hexafluoroisopropanol to the supercritical fluid of carbon dioxide. Thus, by the component separation method using a supercritical fluid chromatograph according to the present invention, highly polar, high-molecular-weight components which have conventionally been difficult to separate can be separated with a high level of separation performance.

Due to the aforementioned properties of hexafluoroisopropanol, the component separation method using a supercritical fluid chromatograph according to the present invention can be considered to be effective for the separation of components which contain the amino group and/or amide group. Specifically, the component separation method using a supercritical fluid chromatograph according to the present invention is particularly effective for the separation of peptides or proteins in a sample.

In the component separation method using a supercritical fluid chromatograph according to the present invention, a mixture prepared by mixing hexafluoroisopropanol with various kinds of conventionally used organic solvents can be used as the modifier.

For example, the modifier may contain methanol or ethanol as the organic solvent. The modifier may further contain isopropanol. The modifier may contain, in addition to methanol or ethanol, water. The modifier may contain, in addition to methanol or ethanol, formic acid.

By appropriately mixing various organic solvents other than hexafluoroisopropanol in this manner, the eluting power can be controlled.

### Advantageous Effects of the Invention

With the component separation method using a supercritical fluid chromatograph according to the present invention, it is possible to separate, with a high level of separation performance, peptides, proteins or other highly polar, high-molecular-weight compounds which have been difficult to separate with a high level of separation performance by conventional supercritical fluid chromatographs. Therefore, a qualitative or quantitative determination of those compounds in a sample can be performed with a high level of throughput and high level of accuracy. Furthermore, those compounds in a sample can be efficiently purified to a high degree of purity.

### BRIEF DESCRIPTION OF DRAWINGS

FIG. 1 is a schematic configuration diagram of the main components of one embodiment of a supercritical fluid chromatograph for carrying out the component separation method according to the present invention.

FIGS. 2A and 2B are measured examples of the chromatograms with and without hexafluoroisopropanol added to the modifier, respectively.

### DESCRIPTION OF EMBODIMENTS

One embodiment of the component separation method according to the present invention is hereinafter described.

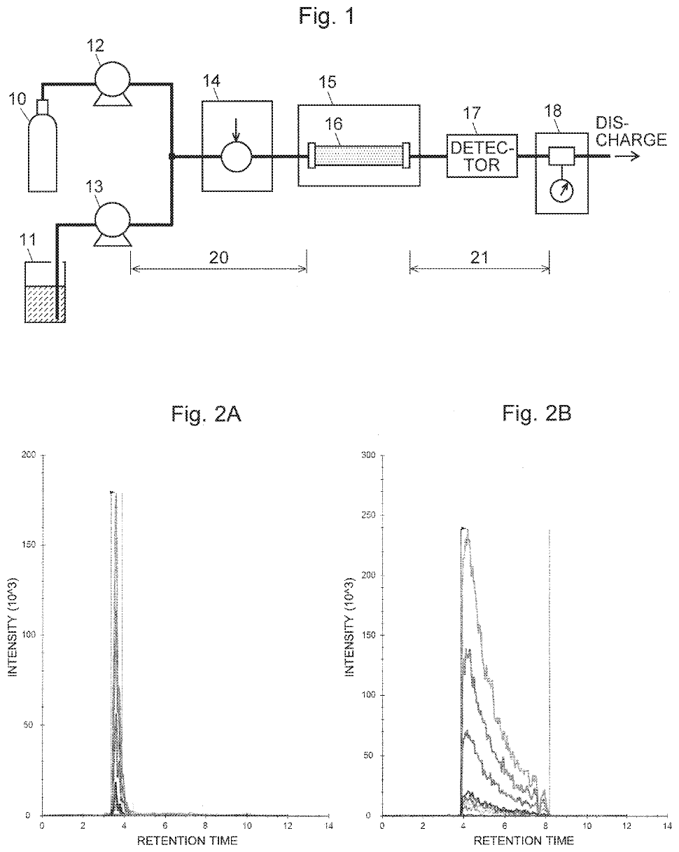

FIG. 1 is a channel configuration diagram of one embodiment of the supercritical fluid chromatograph (SFC) for carrying out the component separation method according to the present invention.

This SFC includes a liquid CO₂ cylinder 10, modifier container 11, CO₂ pump 12, liquid delivery pump 13, autosampler 14, column oven 15, column 16, detector 17 and back pressure regulator 18. The liquid CO₂ cylinder 10 is the supply source of the liquid CO₂ as the supercritical fluid. The modifier container 11 is a container holding a characteristic modifier (which will be described later) to be added to the supercritical fluid.

The basic operation of this SFC is as follows: The back pressure regulator 18 regulates the pressure of the mobile phase at a higher level than the critical pressure (7.4 MPa) for CO₂, e.g. at 10 MPa, in an inlet channel 20 between the CO₂ and liquid delivery pumps 12 and 13 and the inlet port of the column 16, in the column 16, as well as in an outlet channel 21 between the outlet port of the column 16 and the back pressure regulator 18. The CO₂ pump 12 draws the liquid CO₂ from the liquid CO₂ container 10 and delivers it to the inlet channel 20. The liquid delivery pump 13 draws the modifier from the modifier container 11 and delivers it to the inlet channel 20. The liquid CO₂ and the modifier are mixed with each other within the inlet channel 20 and fed to the column 16 as the mobile phase. The autosampler 14 provided in the inlet channel 20 injects a liquid sample into the mobile phase at a predetermined timing.

The column oven 15, with the column 16 contained inside, is regulated at a temperature which exceeds the critical temperature (31° C.) for CO₂ that is the main component of the mobile phase, e.g. at approximately 40° C. Therefore, the mobile phase passing through the column 16 changes to the supercritical state. The sample carried by the flow of the mobile phase and introduced into the column are separated into individual components due to the interaction with the stationary phase provided on the inner wall of the column 16. Those components exit the column 16 at different points in time and flow into the outlet channel 21. The detector 17 provided in the outlet channel 21 is an ultraviolet visible spectrometric detector (UV detector) or similar device, which detects the components separated from each other in the column 16 and contained in the mobile phase as in the supercritical state. After the component detection process, the mobile phase is collected into a collection container or the like (not shown) via the back pressure regulator 18.

As the detector 17, various optical detectors used in liquid chromatographs may also be used, such as a multi-wavelength detector using a photodiode array (PDA), optical rotation detector, circular dichroism detector, fluorescent detector, refractive index detector or evaporative light scattering detector. A mass spectrometer using an atmospheric pressure ion source, such as the electrospray ion source, may also be used as the detector 17.

Modifiers used in conventionally and commonly used SFCs contain methanol, ethanol, isopropyl alcohol, acetonitrile or the like as the main component to which an appropriate amount of formic acid, acetic acid, ammonium formate, ammonium acetate, diethylamine or the like is added as needed. By comparison, in the component separation method according to the present invention, when the sample component to be separated and analyzed is a highly polar biopolymer compound, such as a peptide or protein, a modifier with hexafluoroisopropanol (hexafluoro-2-propanol) added is used.

Hexafluoroisopropanol (chemical formula: (CF₃)₂CHOH; CAS Number: 920-66-1) is a kind of fluoroalcohol. It exhibits a high degree of polarity and high degree of acidity. Hexafluoroisopropanol is miscible with various solvents, such as water, methanol, hexane or isopropanol. In general, hexafluoroisopropanol is used as a solvent for resin materials, such as polyethylene terephthalate (PET), polyamide or polyvinyl alcohol, although peptides and proteins, which are highly polar, high-molecular compounds, can also be dissolved in it. Hexafluoroisopropanol is optically transparent within the ultraviolet wavelength region and does not affect the detection in an UV detector or similar device. Furthermore, hexafluoroisopropanol also does not affect the detection in a mass spectrometer provided with an electrospray ion source, since it does not significantly inhibit ionization in an electrospray ion source.

The effect obtained when hexafluoroisopropanol is added as the modifier is hereinafter described based on the results of actual measurements.

FIG. 2B is a chromatograph obtained by performing a measurement on a peptide-containing sample with an SFC using a mixed liquid of methanol, isopropanol, water and formic acid as the modifier, which is a conventionally and commonly used modifier. As can be seen, a considerable amount of peak tailing has occurred, causing the peak to be broadened from the beginning to the end point. The peak waveform is also unsatisfactory; even the peak top is difficult to locate when the component con-centration is low. Such a low level of separation performance deteriorates the performance of the quantitative determination. A fractionation and purification of the peptide based on such a detection signal will only yield a product with a low degree of purity.

By comparison, FIG. 2A is a chromatogram obtained by performing a measurement on the same sample with the SFC using a mixed liquid prepared by adding an appropriate amount of hexafluoroisopropanol to the mixed liquid of methanol, isopropanol, water and formic acid. A comparison with FIG. 2B evidently shows that satisfactory peaks having sharp waveforms, with each peak having a narrow width from the beginning to the end point, have been obtained. Accordingly, a high level of quantitative determination performance can be achieved. Furthermore, a fractionation and purification of the peptide based on this detection signal will yield a product with a high degree of purity.

Thus, in the previously described SFC, the separation performance for peptides can be dramatically improved by adding hexafluoroisopropanol as the modifier.

Given the effectiveness of hexafluoroisopropanol for peptides, it is easy to conceive the idea that the technique will also produce a sufficient effect for improving the separation performance for proteins, which have even higher molecular weights, as well as other highly polar components which are neither peptides nor proteins yet include the amino group and/or amide group.

In the measured examples mentioned earlier, hexafluoroisopropanol is added to the mixed liquid of methanol, isopropanol, water and formic acid. It is not always necessary to use all of those solvents other than hexafluoroisopropanol. The selection of those solvents can be appropriately changed.

In the previously described SFC, the amount of modifier mixed in the supercritical fluid does not always need to be constant, but may be changed with the passage of time from the point of injection of the sample; i.e. a gradient elusion may be performed.

The previously described embodiment is a mere example of the present invention, and any change, addition or modification appropriately made within the spirit of the present invention will evidently fall within the scope of claims of the present ap-plication.

### REFERENCE SIGNS LIST

10 . . . Liquid CO₂ Cylinder
11 . . . Modifier Container
12 . . . CO₂ Pump
13 . . . Liquid Delivery Pump
14 . . . Autosampler
15 . . . Column Oven
16 . . . Column
17 . . . Detector
18 . . . Back Pressure Regulator

1-7. (canceled)

8. A component separation method using a supercritical fluid chromatograph, comprising steps of:
injecting a sample into a mobile phase containing a supercritical fluid and a modifier; and
introducing the mobile phase with the sample into a column so as to make components in the sample separate from each other while passing through the column,
wherein carbon dioxide is used as the supercritical fluid; and the modifier contains hexafluoroisopropanol.

9. The component separation method using a supercritical fluid chromatograph according to claim 8, wherein a target component in the sample comprises an amino group and/or amide group.

10. The component separation method using a supercritical fluid chromatograph according to claim 9, wherein the target component in the sample is a peptide or protein.

11. The component separation method using a supercritical fluid chromatograph according to claim 8, wherein the modifier contains methanol or ethanol as an organic solvent.

12. The component separation method using a supercritical fluid chromatograph according to claim 11, wherein the modifier further contains isopropanol.

13. The component separation method using a supercritical fluid chromatograph according to claim 11, wherein the modifier further contains water.

14. The component separation method using a supercritical fluid chromatograph according to claim 11, wherein the modifier further contains formic acid.
